(19) United States
(12) Patent Application Publication
(10) Pub. No.: US 2008/0140019 A1
(43) Pub. Date: Jun. 12, 2008

(54) Novel Device

(76) Inventors: Jacques Thilly, Rixensart (BE); Christian Vandecasserie, Rixensart (BE)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/720,561

(22) PCT Filed: Nov. 30, 2005
(86) PCT No.: PCT/EP05/12949

§ 371 Date: May 31, 2007

(30) Foreign Application Priority Data
Dec. 2, 2004 (GB) 0426479.2

(51) Int. Cl. A61M 5/315

(52) U.S. Cl. 604/218

(57) ABSTRACT

A process for filing a syringe in which a longitudinal tubular syringe barrel has a first end closed by a movable plunger and a connection suitable for an injection needle at its second end, this second end of the bore being closed by a penetrable closure, and the syringe is filled by passing a filling needle through the penetrable closure and filing the barrel with a liquid via the needle, then withdrawing the filing needle from the closure. Suitably a penetrator is attached to the second end of the barrel to penetrate and provide communication through the closure.

This invention relates to syringes and to processes for filling syringes.

Syringes, particularly hypodermic syringes, are well known devices for delivery of medicaments by injection through the skin, normally using a hollow injection needle attached to the syringe.

Typically syringes comprise a longitudinal tubular syringe barrel to contain a liquid to be injected, having first and second longitudinally opposite disposed open ends. One end, herein termed the “first end”, is open to receive a plunger to be moved along the barrel towards the opposite second end at which there is a connection suitable for an injection needle. The barrel incorporates a plunger to be driven from the first end toward the second end to thereby expel the liquid from the syringe through the needle. U.S. Pat. No. 3,884,229 discloses a syringe which incorporates a cartridge of liquid medicament sealed with a penetrable closure, the syringe including a penetrator with a needle which penetrates the closure and provides a conduit through the penetrator to a connection for an injection needle at an opposite end of the penetrator. Similar syringes, in which a penetrator is caused to penetrate a penetrable closure to provide communication between the interior of the barrel and an injection needle are disclosed in EP-A-0239673, EP-A-0276160, U.S. Pat. No. 3,989,044, U.S. Pat. No. 4,227,528, U.S. Pat. No. 4,196,732 and WO-A-88/00478.

When filling the liquid into the syringe it is important to avoid contamination of the liquid contents, in particular to maintain sterility. This can be difficult. Normally syringes are filled via one of the open ends of the barrel, either via the open first end before the plunger is inserted into the barrel, or via the open second end before the attachment of the above-mentioned connection for a needle or the penetrable closure. This means that the syringe barrel has to remain open at least at one of these ends until the syringe has been filled, allowing the possibility of contamination of the syringe whilst the syringe awaits filling.

WO-A-2003/028785 discloses a process for filling a syringe in which the plunger is made of a penetrable and fusible material, and the syringe is filled by closing the open first end with the plunger then passing a filling needle through the plunger, filling the syringe barrel via the needle, withdrawing the needle then sealing the residual puncture hole by thermal sealing. Such a plunger is usually relatively thick, requiring considerably puncturing force to drive the filling needle through the plunger. Additionally, because the plunger is intended to be relatively slideably moveable within the barrel, some way must be devised to prevent movement of the plunger under the force applied in driving the filling needle through the plunger.

It is an object of this invention to provide a novel construction of syringe, and a novel process for filling and assembling syringes based on this novel construction of syringe which among other things addresses the problems outlined above. Other objects and advantages of this invention will be apparent from the following description.

According to this invention a syringe is provided comprising a longitudinal tubular syringe barrel defining an internal bore to contain a liquid to be injected, having first and second longitudinally opposite disposed ends, incorporating a plunger to be driven along the bore in a first end toward the second end direction to thereby expel the liquid from the syringe via the second end, wherein:

the second end of the bore is closed by a penetrable closure, adjacent the closure is a penetrator which incorporates a conduit, the penetrator at least partly penetrating the closure such that the conduit provides fluid communication through the closure.

The syringe barrel may be made of conventional materials such as glass or preferably a transparent plastics material, such as known COC polymers. The plunger may be generally conventional, e.g. being connected to an operating shaft for operation by the user.

The penetrable closure may for example comprise a closure wall across the bore, e.g. a disc shaped closure wall of comparable diameter to the diameter of a cylindrically tubular barrel. Alternatively the penetrable closure may for example comprise a convex, in the direction first end toward second end, closure wall across the bore, e.g. a convex dome shaped closure wall. Alternatively such a closure may comprise a plug or cap closing a nozzle outlet at this second end of the barrel. Such a closure wall is preferably made of a material which is easily penetrated by puncturing, e.g. an elastomer material. The penetrable closure may for example include a weakened e.g. puncturable weakened region of such a wall. For example the closure may comprise a penetrable region which is thinned relative to adjacent parts of the closure. Such a region may comprise an integrally thinned region. For example the uppermost part of a convex closure wall may be thinned relative to adjacent, e.g. peripheral parts of the closure wall. Such a region may additionally or alternatively comprise an aperture through the closure, closed with a thin penetrable e.g. puncturable, membrane. Such a region may additionally or alternatively comprise a previously-formed puncture hole, optionally partly sealed e.g. by fusing the material of the closure adjacent to the puncture hole, or sealing the puncture hole in some other way to leave a residual weakened region of the closure. If the closure is wholly or partly made of an elastic material such a puncture hole may be closed prior to penetration by the natural elasticity of the closure causing the elastic material around the puncture hole to come together and close the hole, but leaving the hole easily subsequently re-opened by the penetrator. Such a puncture hole may be closed after the puncture has been made by for example heat sealing e.g. by directing a laser beam at the puncture site, such a so-formed closure then being easily subsequently penetrated. For example alternatively or additionally such a puncture hole may be closed by a thin penetrable membrane prior to penetration. Suitably the closure may include a skirt part which when the closure is in place fits in a plug-like manner into the barrel. Such a skirt part may help to stabilise the closure when in place, and may also facilitate sealing contact between the closure and the barrel by for example providing an increased areas of contact between the barrel and the closure.

For example such a previously-formed formed puncture hole may have been formed by a needle. Such a needle may be a hollow filling needle which has been passed through the closure and via which the liquid has been introduced into the barrel, and the needle then subsequently withdrawn to leave the residual puncture hole. This has the advantage that the empty syringe can be provided in a sterile (the term “sterile” herein includes any level of reduced contamination with undesirable contaminants such as micro-organisms relative to ambient, and in particular contamination reduced to a medically acceptable state) with its interior protected by contamination by the plunger closing the first end and the closure closing the second end. For example the syringe barrel, plunger and closure may be made in a sterile state in a sterile environment and assembled in a sterile environment. WO-A-2005/005128 discloses a process in which vials and their elastomer closures are made in such a way, and this disclosed process may be adapted. The interior of the barrel of the syringe thereby can remain sterile while awaiting filling by the above mentioned procedure, so that advantageously the syringe may only require sterilisation of its exterior e.g. by radiation, prior to filling. Preferably the filling needle used for such a filling procedure has a pyramidal point, as such a point is found to reduce the risk of formation of particles of the closure material during penetration, which may contaminate the interior of the syringe. Particularly suitable filling needles are for example disclosed in WO-A-2004/096114.

For example such a needle may be passed through the closure. The elastic nature of the closure can cause the material of the closure to close when the needle has been withdrawn, to thereby close the residual needle hole to some extent. This offers the advantage that after introducing the liquid into a syringe barrel using a filling needle there is much less opportunity for contamination to enter the syringe than would be the case if after a liquid has been introduced into the barrel, the syringe remains open at the first or second end awaiting closure by insertion of the plunger into the open first end or application of a closure to the open second end of the barrel. Also advantageously after filling using such a filling needle and leaving a closed puncture hole the syringe may be inspected through its transparent wall for particles, with less threat of contamination than would be with known syringes.

The penetrable closure, e.g. such a wall, may be held adjacent to the second end of the barrel by a clamp part, e.g. a collar around the second end of the barrel and bearing upon the closure to press the closure against the second end, with a pressure sufficient to form a liquid tight seal between the barrel and the closure. Such a clamp part may snap fit onto the second end of the barrel. For example the second end of the barrel may comprise a sealing flange against which the closure is pressed by the clamp part, typically surrounding the second end of the barrel, and the surface of such a flange in contact with the closure may incorporate a ring-shaped sealing ridge to enhance the seal between the flange and closure. Such a clamp part may also be made in a sterile environment as described above.

The penetrator provides communication through the closure, i.e. between the interior and exterior of the barrel, by providing a conduit through the closure when the penetrator at least partly penetrates the closure. The penetrator may comprise a generally tubular member along the tubular bore of which the liquid may flow, having an end adapted to at least partly penetrate the penetrable closure. Typically such an end may be generally pointed, e.g. being a generally conical end with a hole adjacent its apex. The penetrator may, for example integrally, comprise a connector for a hypodermic needle. For example the opposite end of such a tubular penetrator may, for example integrally, comprise a connector for a hypodermic needle. By means of such a connector liquid expelled from the syringe via the second end of the barrel may be caused to flow through a needle connected thereto, e.g. for injection of the liquid into a patient. Such a connector may for example be shaped as a male member to fit into the female connector of a typical hypodermic needle. The connector of the penetrator may be provided with a safety closure prior to connection to a needle. Such a safety closure may be of a generally known type e.g. a cap over a needle connector nozzle.

The penetrator may be adapted to attach to the syringe. Such attachment may be by means of a snap-fit engagement between the penetrator and the barrel, or between the penetrator and the above-mentioned clamp part holding the closure in place. When the penetrator engages with the clamp part this engagement may usefully be employed to cause the penetrator to compress the closure against the barrel to thereby enhance the seal between the closure and the barrel.

The penetrator may comprise a flange surface which bears upon the surface of the closure opposite to that which bears upon the above-mentioned sealing flange of the barrel, and such a flange surface in contact with the closure may incorporate a ring-shaped sealing ridge to enhance the seal between the flange surface and closure.

The plunger may be generally conventional, i.e. an elastomeric cylinder, preferably slightly greater in diameter than the internal diameter of the barrel, so that the elasticity of the plunger causes it to be expansively compressed against the inner surface of the bore of the barrel in a conventional manner. Preferably a backstop is provided at the first end of the barrel to resist any tendancy of the plunger to be removed from the barrel via the first end. Such a backstop may comprise an abutment to abut against the plunger if the plunger tends to leave via this first end. Such an abutment may comprise a flange protruding slightly inside the inner diameter of the barrel. The insertion of the plunger into the barrel via the first open end whilst the closure at the second end is in place may cause overpressure in the barrel, and the backstop can both retain the plunger in place against this overpressure, and maintain the overpressure as an additional protection against contamination ingress. Furthermore such a backstop may serve a further role in that introduction of a liquid into the syringe via a filling needle inserted through the closure at the second end may increase this overpressure. Such a backstop may also comprise a conical shaped entrance, and the plunger may have a corresponding conical shape tip to help guide and compress the plunger during insertion into the first open end of the syringe. Such a backstop may be attachable to the barrel adjacent the first open end by for example a snap-fit connection. The end of the plunger facing away from the first open end of the syringe may include a central concavity to help the compression and deformation of the plunger during insertion into the barrel.

A specific preferred construction of syringe of this invention therefore comprises a longitudinal tubular syringe barrel defining an internal bore to contain a liquid to be injected, having first and second longitudinally opposite disposed ends, incorporating a plunger to be driven along the bore in a first end toward the second end direction to thereby expel the liquid from the syringe via the second end, wherein:

the second end of the bore is closed by a penetrable closure in the form of a closure wall across the bore made of an elastomer material, being penetrable at a previously-formed puncture hole providing a weakened region at which the closure may be penetrated,

the closure being held adjacent to a flange surface at the second end of the barrel by a clamp part at the second end of the barrel and bearing upon the closure to press the closure against the second end, with a pressure sufficient to form a liquid tight seal between the barrel and the closure,

adjacent the closure being a penetrator which incorporates a conduit, the penetrator at least partly penetrating the closure such that the conduit provides fluid communication between the interior and the exterior of the barrel, the penetrator comprising a generally tubular member along the tubular bore of which the liquid may flow, having an end adapted to at least partly penetrate the penetrable closure, the opposite end of the penetrator comprising a connector for a hypodermic needle.

By “at least partly penetrates” herein is included passage of the penetrator from an outer side of the closure at least partly to an inner side, e.g. puncturing and physically disrupting the closure, expansion of an already existing hole in the closure by means of the penetrator, disruption of a weakened area of the closure by the penetrator to create an opening through the closure, and any other kind of penetration of the closure by the penetrator that provides the fluid communication.

Adjacent to open first end of the

The invention further provides a process for providing a syringe filled with a liquid, comprising the steps of:

providing a tubular syringe barrel defining an internal bore to contain a liquid to be injected, the barrel having first and second longitudinally opposite disposed ends, the first end being open to receive a plunger to be moved along the barrel towards the opposite second end to thereby expel the liquid from the syringe via the second end, the barrel incorporating such a plunger, at the second end being a connection suitable for connecting an injection needle to the barrel, the second end of the bore being closed by a penetrable closure,

passing a filling needle through the penetrable closure and filling the barrel with a liquid via the needle,

then withdrawing the filling needle from the closure.

Preferably the process includes the further step of attaching a penetrator which incorporates a conduit to the barrel adjacent the second end, such that the penetrator at least partly penetrates the closure such that the conduit provides fluid communication through the closure. Suitably this step may involve effecting a snap-fit engagement between the penetrator and the barrel, or between the penetrator and a clamp part holding the closure in place.

Preferably after withdrawing the filling needle from the closure, e.g. between the step of withdrawing the filling needle from the closure and attaching the penetrator, there is the further step of closing the residual puncture hole by for example heat sealing, i.e. to melt or soften the material of the closure by heat adjacent to the puncture hole so that the material fuses to close the hole. Heat sealing may for example be by directing a laser beam at the puncture site, and/or closing the puncture hole by a thin penetrable membrane, or by applying a sealing substance to the closure in the vicinity of the puncture hole.

Suitably the syringe is provided for the process of this invention with its interior, i.e. the volume bounded by the barrel, the plunger and the closure, in a sterile state. Suitably before the filling needle is passed through the closure the outer surface of the closure is sterilised, for example using radiation, e.g. electron beam radiation.

Suitable and preferred features of the syringe, its barrel, its plunger, the penetrable closure, the penetrator, other parts of the syringe, and the way in which these elements may be engaged together are as discussed above.

The process of the invention and the above-mentioned preceding steps are preferably performed in a sterile environment, for example in a downward laminar flow of purified air. Use of such environments is standard in filling and assembly processes for syringes for medicinal uses. Typically syringe barrels, with their plungers and closures in place may be loaded onto a conveyor line and moved into a position adjacent to a filling station at which the filling needle may be passed through the closure and the barrel filled, then adjacent to an attachment station at which the penetrator may be attached. Between the filling station and the attachment station there may be a sealing station at which the residual puncture hole may be closed by for example heat sealing e.g. by directing a laser beam at the puncture site, and/or closing the puncture hole by a thin penetrable membrane, or by applying a sealing substance to the closure in the vicinity of the puncture hole.

The advantages of this process derive from the above-mentioned advantages of the syringe, e.g. in that after introducing the liquid into a syringe barrel using a filling needle there is much less opportunity for contamination to enter the syringe, and the possibility of inspecting the contents of the syringe through a wall made of a transparent wall material for particles, with less threat of contamination than would be with known syringes filling and assembly processes.

The invention will now be described by way of example only with reference to the accompanying drawings.

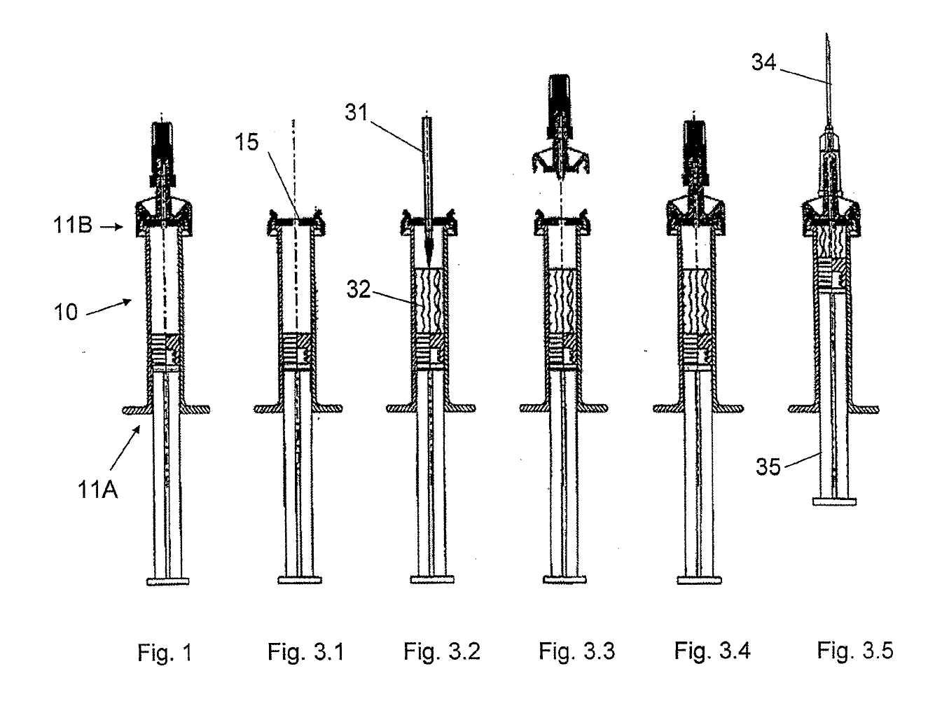

FIG. 1 shows a longitudinal section through a syringe of the invention.

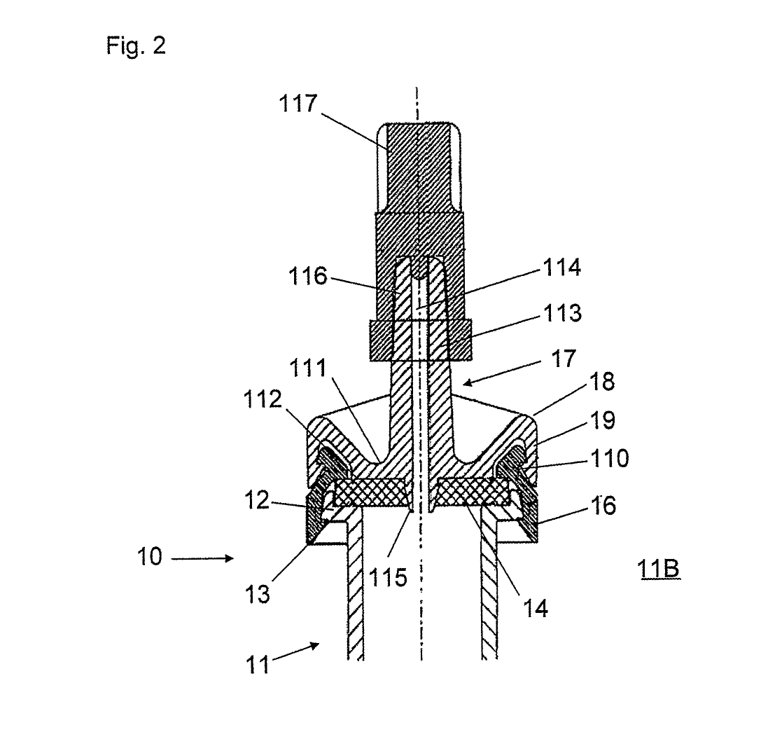

FIG. 2 is an enlargement of the second end of the syringe of FIG. 1

FIG. 3 shows schematically a syringe assembly and filling process of the invention.

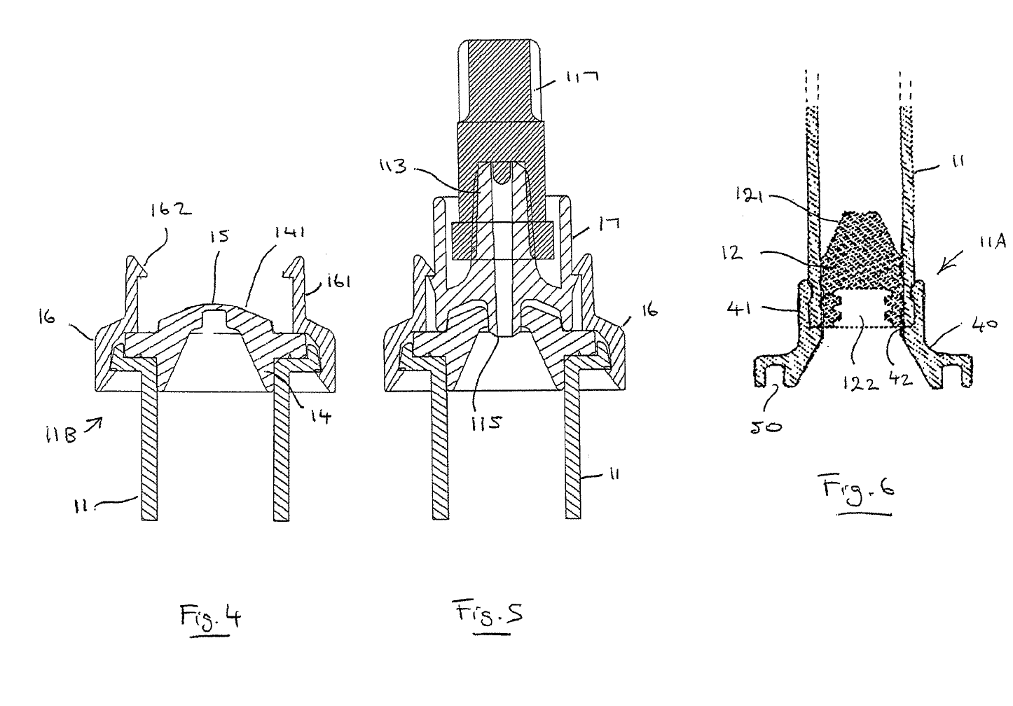

FIGS. 4 and 5 show a longitudinal section through part of another syringe of the invention.

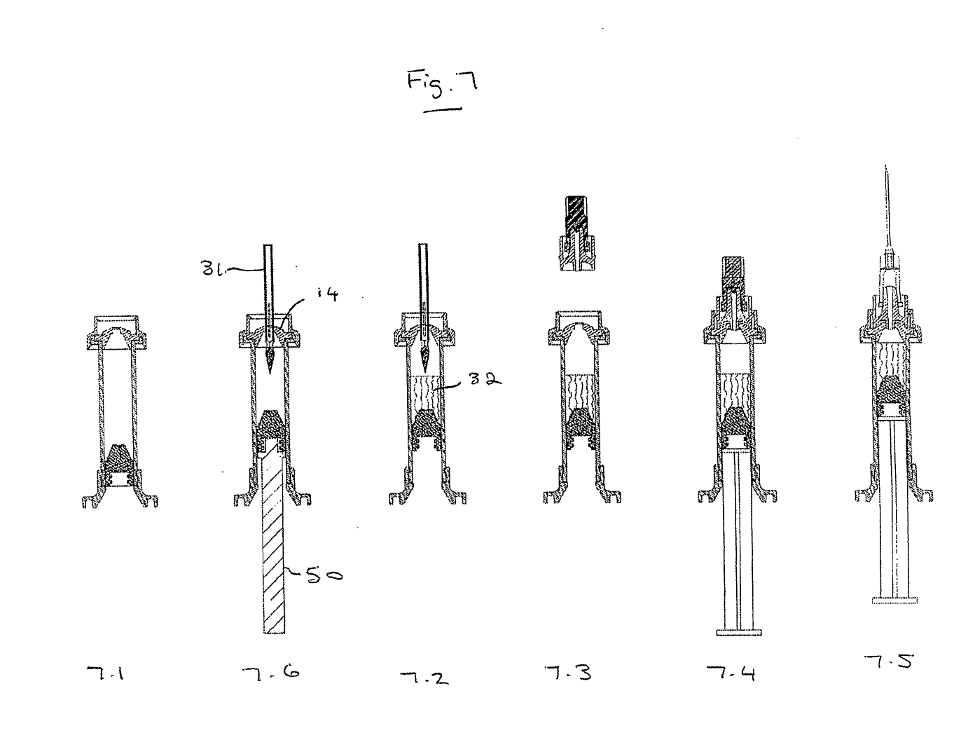

FIG. 6 shows a longitudinal section through part of another syringe of the invention.

FIG. 7 shows schematically a further syringe assembly and filling process of the invention.

10 syringe generally

11 barrel

11A first end of the barrel

11B second end of the barrel

12 plunger

121 conical end of plunger

122 central concavity

13 sealing ridge

14 penetrable closure

141 dome shaped part of closure

142 skirt part

15 integrally thinned region

16 clamp part

161 skirt

162 snap-fit bead

17 penetrator

18 flange

19 skirt part

110 groove

111 flange surface

112 sealing ridge

113 tubular member

114 tubular bore

115 penetrator lower end

116 penetrator opposite end

117 safety closure

31 filling needle

32 liquid

33 residual puncture hole

34 hypodermic needle

35 shaft

40 backstop

41 sleeve

42 flange

43 conical entrance to backstop

44 shaped base

50 tool

Referring to FIGS. 1 and 2 a syringe 10 is shown overall. Syringe 10 comprises a longitudinal tubular syringe barrel 11 to contain a liquid (not shown) to be injected, having first 11A and second 11B longitudinally opposite disposed open ends. Barrel 11 is made of a conventional transparent polymer, and incorporates an internal plunger 12 of generally conventional construction to be driven in a first end 11A toward the second end 11B direction to thereby expel the liquid from the syringe 10 via the second end 11B. The second end 11B of the barrel 11 is integrally formed into a sealing flange 12 surrounding the second end 11B of the barrel 11, and the surface of flange 12 integrally incorporates a ring-shaped knife edge sealing ridge 13.

Second end 11B is closed by a penetrable closure 14, which comprises an elastomer material wall, ca. 1 mm thick, disc shaped to correspond to the circular section of the barrel, and incorporating a central integrally thinned region 15. The closure 14 seats on flange 12, and the ring-shaped sealing ridge 13 enhances the seal between the flange 12 and closure 14.

Closure 14 is held in place adjacent end 11B of the barrel by clamp part 16, in the form of a ring-shaped collar around the second end 11B of the barrel 11, which bears upon the closure 14 to press the closure against the flange 12, and engages in a snap-fit manner with the underside (as seen) of flange 12, to apply a pressure to closure 14 sufficient to form a liquid tight seal between the barrel 11 and the closure 14.

A penetrator 17 is mounted on the barrel 11, by means of a flange 18 which engages in a snap-fit manner with clamp part 16 holding the closure 14 in place. For example a skirt part 19 of flange 18 engages with a corresponding groove 110 around the clamp part 16. The flange 18 of penetrator 17 has a flange surface 111 which bears upon the surface of the closure 14 opposite to that which bears upon the flange 12 and also incorporates a ring-shaped sealing ridge 112 to enhance the seal between the flange surface 111 and closure 12.

The penetrator 17 incorporates a conduit provided by a generally tubular member 113 having a tubular bore 114 along which the liquid may flow. Penetrator 17 has a lower (as seen) end 115 adapted to at least partly penetrate the penetrable closure 12, end 115 being a generally conical member with the bore 114 opening at a hole adjacent the cone apex. The opposite end 116 of penetrator 17 is integrally shaped into the form of a standard male connector for a standard hypodermic needle (not shown). The end 116 is provided with a safety closure 117.

Penetrator 17 is seen to penetrate the closure 14 such that the conduit 114 provides fluid communication between the interior and the exterior of the barrel 11. The closure 14 is penetrable at the thinned region 15 at which the closure may be penetrated. As will be seen in FIG. 3 a previously formed puncture hole has been made through thinned region 15, but which is closed prior to penetration by the natural elasticity of the closure 14 causing the elastomer material around the puncture hole to come together and close the hole, but leaving the hole able to be easily re-opened by the end 115 of the penetrator 17.

Referring to FIGS. 4 and 5 the part adjacent to the second end 11B another syringe 10 of the invention is shown in longitudinal section. Parts corresponding to FIGS. 1 and 2 are numbered correspondingly. In the syringe of FIGS. 4 and 5 the penetrable closure 14 comprises a convex dome shaped closure wall 141, of which the uppermost part includes a penetrable region 15 which is thinned relative to adjacent parts of the domed part 141 of closure 14. This region 15 is an integrally thinned region. The closure 14 also comprises a skirt part 142 which fits plug-like into the bore of barrel 11. Clamp part 16 comprises an upward directed skirt 161 with an inward facing snap fit bead 162 to snap-fit engage a penetrator 17. FIG. 5 shows the part of the syringe as shown in FIG. 4 with the penetrator 17 in place snap fit engaged with the bead 162. In this construction the lower end of the penetrator 17 itself bears upon the closure 14 to compress the closure 14 against the end 11B of barrel 11 additionally to the compression of the closure against the end 11B also being provided by the clamp part 16. In FIG. 5 the penetrator 17 is seen penetrating the domed part 141 of closure 14, prior to which a filling needle (not shown in FIGS. 4 and 5) has been passed through this domed part 141 to create a puncture hole (not shown in FIGS. 4 and 5).

Referring to FIG. 6 some detail of the first end 11A of the syringe 10 is shown. A backstop 40 is provided at the first end 11A of the barrel. Backstop 40 comprises a sleeve part 41 which snap-fit engages with the barrel 11, and has an internal diameter slightly less that the internal diameter of barrel 11, to thereby provide a flange 42 protruding slightly inside the inner diameter of the barrel. FIG. 6 shows how the plunger 12 abuts against the flange 42 to resist any tendancy of plunger 12 to escape from the barrel 11 via its open end 11A. Backstop 40 has a conical shaped entrance 43, and plunger 12 has a corresponding conical shape tip 121. The end of the plunger 12 facing away from the first open end 11B of the syringe 10 includes a central concavity 122 to help the compression and deformation of the plunger 12 during insertion into the barrel 11 via open end 11B, and also to engage a shaft (not shown in FIG. 6, seen as 35 in FIGS. 1 and 3). Backstop 40 also includes a shaped base 44 to assist mounting the barrel 11 onto a conveyor for filling.

Referring to FIG. 3 a process for assembling a syringe is shown schematically.

In FIG. 3.1 a syringe 10 is shown as described above, comprising the longitudinal tubular syringe barrel 11 to contain a liquid to be injected, the second end 11B being closed by the penetrable closure 14 with its thinned central region 15, the closure 14 being held in place on the barrel 11 by the clamp part 16. The interior of the barrel 11, and all surfaces of the barrel 11, plunger 12, and closure 14 which are in contact with the interior of the barrel 11 are sterile. This has been achieved by making and assembling barrel 11, plunger 12, closure 14, and the clamp part 16 in a sterile environment.

In FIG. 3.2 a hollow filling needle 31, having a pyramidal point, is passed through closure 12 and liquid 32 is introduced into the barrel 11 via needle 31. Then the needle is withdrawn, leaving a residual puncture hole 33 through the thinned region 15. Immediately prior to passing the needle 31 through closure 12 the outer surface of the closure 12 and adjacent regions of the clamp part 16 have been sterilised by exposure to a beam of electrons of a known intensity sufficient to inactivate any micro-organisms thereon. The filling needle 31 is provided with external venting grooves (not shown) which allow atmosphere within barrel 11 to vent as liquid 32 is introduced into barrel 11 to thereby displace the atmosphere within.

FIG. 3.3 shows the attaching of the penetrator 17 (as described above) to the syringe 10 by a snap fit attachment of the penetrator 17 with the clamp part 16 (as described above). The conically pointed end 115 of penetrator 17 penetrates the closure 14 at the residual puncture hole 33 through the thinned region 15 such that the conduit 114 provides fluid communication between the interior and the exterior of the barrel 11. The safety closure 117 closes the connector part 113 of penetrator 17. Prior to attaching the penetrator 17 to clamp part 16 the penetrator 17 has been sterilised to inactivate any potentially contaminating micro-organisms thereon.

FIG. 3.4 corresponds to FIG. 1, with the barrel 11 containing the liquid 32.

FIG. 3.5 shows how the syringe 10 may be used. The closure 117 is removed and a standard hypodermic needle 34 is engaged with the connector 116. The plunger 12 may now be driven in the first end 11A toward the second end 11B direction to thereby expel the liquid 32 from the syringe via the second end 11B through the conduit 114 and needle 34 by hand pressure on the conventional shaft 35.

Referring to FIG. 7 a process for filling and assembling a syringe is shown schematically using the barrel of FIGS. 4, 5 and 6. It will be noted immediately that FIGS. 7.1, 7.2, 7.3, 7.4 and 7.5 correspond generally respectively to FIGS. 3.1, 3.2, 3.3, 3.4 and 3.5. However the additional step 7.6 is included after the filling needle 31 has been passed through the closure 14 but before any liquid 32 has been introduced into the barrel 11 in step 7.2, in which the plunger 12 is moved along the barrel 11 toward the second end 11B to its final position for use by a tool 50 pushing the plunger 12 upwards, allowing atmosphere within barrel 11 to escape via the venting grooves (not shown) of the needle 31. It is noted that as it is pushed upwards the plunger 12 moves only along the sterile interior of the barrel, minimising the risk to sterility.

The process schematically illustrated in FIGS. 3 and 7 is preferably performed in a sterile environment, for example in a downward laminar flow of purified air. Preferably the syringes 10 are mounted for the process illustrated in FIG. 3 and 7 on a conveyor (not shown) which conveys the syringes 10 sequentially past a filling station (not shown) at which the needle is passed through the closure 14, and an attachment station (not shown) at which the penetrator 18 is attached by snap fitting to the clamp part 16.

We claim:

1. A process for providing a syringe filled with a liquid, comprising the steps of:
providing a tubular syringe barrel defining an internal bore to contain a liquid to be injected, the barrel having first and second longitudinally opposite disposed ends, the first end being open to receive a plunger to be moved along the barrel towards the opposite second end to thereby expel the liquid from the syringe via the second end, the barrel incorporating such a plunger, at the second end being a connection suitable for connecting an injection needle to the barrel, the second end of the bore being closed by a penetrable closure,
passing a filling needle through the penetrable closure and filling the barrel with a liquid via the needle,
then withdrawing the filling needle from the closure.

2. A process according to claim 1 characterised by the further step, after withdrawing the filling needle from the closure, of attaching a penetrator which incorporates a conduit to the barrel adjacent the second end, such that the penetrator at least partly penetrates the closure such that the conduit provides fluid communication through the closure.

3. A process according to claim 2 characterised in that the penetrator comprises a generally tubular member along the tubular bore of which liquid may flow, having an end adapted to at least partly penetrate the penetrable closure, and comprising a connector for a hypodermic needle.

4. A process according to claim 2 characterised by effecting a snap-fit engagement between the penetrator and the barrel, or between the penetrator and a clamp part holding the closure in place on the barrel.

5. A process according to claim 1, characterised in that after withdrawing the filling needle from the closure there is the further step of closing the residual puncture hole.

6. A process according to claim 2, characterised in that between the step of withdrawing the filling needle from the closure and attaching a penetrator there is the further step of closing the residual puncture hole.

7. A process according to claim 5 characterised in that the further step of closing the residual puncture hole is achieved by heat sealing.

8. A process according to claim 1 characterised in that the syringe is provided before with its interior in a sterile state.

9. A process according to claim 8 characterised in that before the filling needle is passed through the closure the outer surface of the closure is sterilised.

10. A process according to claim 1 characterised in that a filling needle is passed through the closure, and the plunger is then moved along the barrel toward the second end toward its final position for use, allowing atmosphere within the barrel to vent.

11. A process according to claim 1 characterized by being performed by loading syringe barrels, with their plungers and closures in place, onto a conveyor line and moved the barrels into a position adjacent to a filling station at which the filling needle is passed through the closure and the barrel filled, then moving them adjacent to an attachment station at which the penetrator is attached.

12. A process according to claim 11 characterised in that between filling station and the attachment station there is a sealing station at which the residual puncture hole is closed.
